(19) United States
(12) Patent Application Publication
(10) Pub. No.: US 2018/0325812 A1
(43) Pub. Date: Nov. 15, 2018

(54) OCULAR COMPOSITIONS

(71) Applicant: THE QUEEN'S UNIVERSITY OF BELFAST, Belfast Antrim (GB)

(72) Inventors: Raghu Raj Singh THAKUR, Belfast Antrim (GB); David JONES, Belfast Antrim (GB); Chirag GUJRAL, Belfast Antrim (GB)

(21) Appl. No.: 15/774,828

(22) PCT Filed: Nov. 10, 2016
(86) PCT No.: PCT/EP2016/077269

§ 371 Date: May 9, 2018

(30) Foreign Application Priority Data
Nov. 10, 2015 (GB) 1519811.2

(51) Int. Cl. A61K 9/00; A61K 47/34; A61K 47/32; A61K 47/22; A61K 47/18; A61K 9/51; C07K 16/22; A61N 5/06

(52) U.S. Cl. CPC A61K 9/0048; A61K 2039/54; A61K 9/0024; A61K 47/34; A61K 47/32; A61K 47/22; A61K 47/183; A61K 9/5138; A61K 9/5146; A61K 9/5153; A61K 9/5123; C07K 16/22; A61N 5/062; C07K 2317/24; C07K 2317/76; A61K 9/0051

(57) ABSTRACT

The invention provides an ocular composition comprising: 99 to 60% (w/w) of a photopolymerizable composition selected from the group of fragments or monomers consisting of polyalkylene glycol diacrylate and polyalkylene glycol dimethacrylate, wherein the photopolymerizable composition has a molecular weight in the range of 100 to 20,000 Dalton; a biodegradable polymer selected from the group consisting of aliphatic polyester-based polyurethanes, polylactides, polycaprolactones, polyorthoesters and mixtures, copolymers, and block copolymers thereof; a photoinitiator; and a therapeutic agent. The composition can be used to form an ocular implant and an in situ ocular implant.

### BACKGROUND OF THE INVENTION

Chronic retinal diseases are the leading contributor to visual impairment and blindness worldwide. Loss of sight has a major personal impact on people's daily lives and has a profound economic impact on individuals, families, support agencies, society and the state. The World Health Organization estimates that globally about 285 million people are visually impaired, of which 39 million are blind and 246 million have low vision. Diseases that originate in the posterior segment (PS) or back of the eye lead to permanent loss of vision if left untreated and account for the majority of blindness, such as in age-related macular degeneration (AMD), diabetic retinopathy (DR), diabetic macular edema (DME), cytomegalovirus (CMV) retinitis, retinitis pigmentosa, uveitis and glaucoma. The PS of the eye, which includes the retina, choroid, and vitreous body, is difficult to access due to the recessed location within the orbital cavity. Therefore, drug delivery to the PS of the eye has remained one of the most challenging tasks for pharmaceutical scientists and retina specialists.

Multiple approaches have been used to deliver drugs to the PS of the eye such as systemic, topical, periocular (or transscleral) and intravitreal. Topical (e.g. eye drops) and systemic (e.g. oral tablets) routes result in low or sub-therapeutic drug levels due to multiple ocular barriers, requiring administration of unnecessarily high concentrations of drug that causes drug-related toxicity and producing low treatment efficacy. The periocular route involves injections on the outer surface of the eye. Depending upon their area of injection they are termed as subconjunctival, peribulbar, subtenon and retrobulbar injections. Following injection, transient diffusion of drug occurs across the the large white structural sheath around the circumference of the eye. Drug diffusion across the scleral membrane is dependent upon drug's solubility, molecular weight/molecular radius, charge and polarity. However, this method has shown low intraocular bioavailability due to a delay in drug diffusion through the sclera, systemic clearance and loss of drug before reaching the target tissues (e.g. retina/choroid). One of the standard treatments to prevent the above chronic conditions is by frequent intravitreal injections (direct injection into the eye) of drug solutions (e.g. ranibizumab, bevacizumab, triamcinolone acetonide and dexamethasone) using traditional hypodermic needles. Intravitreal injections have the advantage of delivering drugs directly to the required site, unlike conventional topical or systemic routes. However, this is not a desirable method of drug delivery for several reasons: the need for frequent injections, significant tissue trauma, short half-lives of injected drugs, uncomfortable and painful to patients, requires professional training, causes rise in intraocular pressure (IOP), can result in severe injection-related infections (e.g. endophthalmitis, hemorrhage, and cataract), mechanical injury to the lens and retina, high drug-induced toxicities, and higher costs. Furthermore, post intravitreal injection patients may require hypotensive treatment to maintain normal IOP, and may even undergo glaucoma surgery. These risks are dependent upon the needle type, where lower gauge needles causes higher damage to the eye. Therefore, reducing the dosing frequency is the greatest, realistic unmet need in treating these diseases. Hence, there is a great need to develop long-acting controlled release delivery systems. To this end, limited controlled release intraocular implants have been engineered that are either injected with hypodermic needles, or surgically sutured to the eye, yet these methods of administration are highly invasive and cause excessive tissue trauma. For example: Vitrasert® (ganciclovir implant), Retisert® (fluocinolone acetonide implant), Iluvien® (fluocinolone acetonide implant), and Ozurdex™ (dexamethasone implant) that are commercially available to treat cytomegalovirus retinitis, uveitis, DME and macular edema/uveitis, respectively. Other products under development include; I-Vation™ for AMD and DME; Rh CNTF for retinal pigmentation and AMD; Visulex® for uveitis; and Verisome™ for AMD and DR indications. Vitrasert®, Retisert®, I-Vation™ and Rh CNTF are non-biodegradable and surgically implanted in the eye, with attendant higher risks for infections, higher cost of administration, increased IOP and low patience compliance. Also, these implants require a secondary surgical procedure to either remove or replace with a new implant. Iluvien® (non-biodegradable) & Ozurdex® (biodegradable) are injected into the eye by using 25G and 22G needles, respectively, a procedure that takes ˜20 minutes to accomplish, causing considerable pain/discomfort and significant morbidity (subconjunctival haemorrhage, vitreous leak and increased IOP). Alternatively, non-invasive devices for sustained transscleral delivery such as iontophoresis, electroporation, electrophoresis, and photoacoustic could avoid surgical intervention to significantly improve patient compliance. However, wear time of these devices (e.g. 5 to 20 min for iontophoresis), degree of discomfort to patients, potential for adequate sustained release, costs and safety of long-term application are yet to be established. Furthermore, no long-acting or controlled release protein/peptide-based therapies have gained approval in an implant system to date.

Therefore, there is a need for new and improved systems for ocular delivery of therapeutic agents.

### SUMMARY OF THE INVENTION

The present invention provides ocular compositions that can be administered to the eye in various forms to achieve controlled release of a therapeutic agent (or drug). The invention allows the flexibility to administer a range of small and large drug molecules including proteins, peptides and gene therapeutics, and maintain their activity for a controlled period of time. The invention also provides methods of treating a number of eye diseases comprising administering the ocular compositions of the invention to a subject in need thereof.

According to a first aspect of the invention, there is provided an ocular composition comprising:
i) 99 to 60% (w/w) of a photopolymerizable composition selected from the group of fragments or monomers consisting of polyalkylene glycol diacrylate and polyalkylene glycol dimethacrylate, wherein the photopolymerizable composition has a molecular weight in the range of 200 to 20,000 Dalton;
ii) a biodegradable polymer selected from the group consisting of aliphatic polyester-based polyurethanes, polylactides, polycaprolactones, polyorthoesters and mixtures, copolymers, and block copolymers thereof;
iii) a photoinitiator; and
iv) a therapeutic agent.

Optionally, the composition is used to form an ocular implant or the composition is used to coat an ocular implant.

Optionally, the implant is an in situ formed ocular implant, wherein, further optionally, the photopolymerizable composition has a molecular weight in the range of 200 to 1,000 Dalton.

Alternatively, the implant is a pre-formed ocular implant.

Optionally, the biodegradable polymer is selected from the group of collagen, chitosan, poly(propylene fumarate), lactide/glycolide copolymer (PLGA), polylactic acid (PLA), polyglycolic acid (PGA), polycaprolactone (PCL), lactide/caprolactone copolymer (PLC), poly (L-lactide) (PLLA), and mixtures, copolymers, and block copolymers thereof.

Further optionally, the biodegradable polymer is selected from the group lactide/glycolide co-polymer (PLGA), polylactic acid (PLA), polyglycolic acid (PGA), polycaprolactone (PCL), lactide/caprolactone copolymer (PLC), poly (L-lactide) (PLLA) and mixtures, copolymers, and block copolymers thereof. Still further optionally, the biodegradable polymer is PLGA.

Alternatively, the biodegradable polymer is selected from the group PCL, PLC, PLLA, and mixtures, copolymers, and block copolymers thereof.

Optionally, the photopolymerizable composition is a polyalkylene glycol diacrylate fragment or monomer incorporating diacrylate end units selected from the group comprising polyether fragments or monomers, polyester fragments or monomers, polycarbonate fragments or monomers and mixtures, copolymers, and block copolymers thereof.

Alternatively, the photopolymerizable composition is selected from the group consisting of polyethylene glycol diacrylate, diethylene glycol diacrylate, polyethylene glycol dimethacrylate, diethylene glycol dimethacrylate, polypropylene glycol diacrylate, dipropylene glycol diacrylate, dipropylene glycol dimethacrylate, and polypropylene glycol dimethacrylate.

Optionally, the photopolymerizable composition is polyethylene glycol diacrylate or polyethylene glycol dimethacrylate. Further optionally, the photopolymerizable composition is polyethylene glycol diacrylate or is PLGA.

Optionally, the molar ratio of lactic acid to glycolic acid in the PLGA is 90% lactic acid to 10% glycolic acid, 85% lactic acid to 15% glycolic acid, 75% lactic acid to 25% glycolic acid, 65% lactic acid to 35% glycolic acid, 50% lactic acid to 50% glycolic acid, 35% lactic acid to 65% glycolic acid, 25% lactic acid to 75% glycolic acid, 15% lactic acid to 85% glycolic acid, and 10% lactic acid to 90% glycolic acid.

An optional ocular composition comprises:
i) 79.5 to 59.5% (w/w) polyethylene glycol diacrylate or polyethylenene glycol dimethacrylate; and
ii) 20 to 40% (w/w) PLGA, wherein the molar ratio of lactic acid to glycolic acid in the PLGA is 90% lactic acid to 10% glycolic acid, 85% lactic acid to 15% glycolic acid, 75% lactic acid to 25% glycolic acid, or 50% lactic acid to 50% glycolic acid.

Further optionally, the ocular composition comprises:
i) 69.5% (w/w) polyethylene glycol diacrylate or polyethylene glycol dimethacrylate; and
ii) 30% (w/w) PLGA wherein the molar ratio of lactic acid to glycolic acid in the PLGA is 90% lactic acid to 10% glycolic acid, 85% lactic acid to 15% glycolic acid, 75% lactic acid to 25% glycolic acid, or 50% lactic acid to 50% glycolic acid.

An optional ocular composition of the invention comprises:
i) 95.5 to 84.5% (w/w) polyalkylene glycol diacrylate or polyalkylene glycol dimethacrylate; and
ii) 4 to 15% (w/w) PCL.

Another optional ocular composition of the invention comprises:
i) 79.5 to 94.5% (w/w) polyalkylene glycol diacrylate or polyalkylene glycol dimethacrylate; and
ii) 20 to 5% (w/w) PLLA.

Another optional ocular composition of the invention comprises:
i) 95.5 to 84.5% (w/w) polyalkylene glycol diacrylate or polyalkylene glycol dimethacrylate; and
ii) 4 to 15% (w/w) PLC in which lactic acid to caprolactone is in the range of 90% lactic acid to 10% caprolactone, 80% lactic acid to 20% caprolactone, 70% lactic acid to 30% caprolactone, 60% lactic acid to 40% caprolactone, or 50% lactic acid to 50% caprolactone.

The ocular composition of the invention optionally further comprises a solvent selected from dimethyl sulfoxide, decylmethyl sulfoxide, 2-pyrrolidone, 1-methyl-2-pyrrolidne, N-vinyl-pyrrolidine, N-Methyl-2-pyrrolidone, N-ethyl-pyrrolidone, glycerol formal, glycerol, polyethylene glycol, propylene glycol, benzyl alcohol, benzyl benzoate, ethyl benzoate, triacetin, triethyl citrate, dimethylformamide, dimethylacetamide and tetrahydrofuran. The solvent may be selected from dimethyl sulfoxide, decylmethyl sulfoxide, 2-pyrrolidone, 1-methyl-2-pyrrolidne, N-Methyl-2-pyrrolidone, and glycerol formal.

The ocular composition of the invention optionally further comprises a pore-forming agent. Optionally, the pore-forming agent is selected from polyethylene glycol, maltose, glucose, agarose, mannitol, gelatin, sodium chloride, magnesium carbonate, magnesium hydroxide, potassium chloride, sodium bicarbonate, potassium bicarbonate, and sucrose.

The photopolymerizable composition may be polymerized by irradiating the composition with light at a wavelength of between 230 to 550 nm, between 300 to 525 nm, or between 350 to 490 nm for between 1 second and 60 minutes.

Optionally, the biodegradable polymer is essentially contained within a matrix of the photopolymerizable composition.

The photoinitiator may be selected from a hydroxyketone photoinitiator, an amino ketone photoinitiator, a hydroxy ketone/benzophenone photoinitiator, a benzyldimethyl ketal photoinitiator, a phenylglyoxylate photoinitiator, an acyl phosphine oxide photoinitiator, an acyl phosphine oxide/alpha hydroxy ketone photoinitiator, a benzophenone photoinitiator, a ribityl isoalloxazine photoinitiator, or a phenylglyoxylate photoinitiator or any combination thereof. Optionally, the photoinitiator is 1-[4-(2-hydroxyethoxy)-phenyl]-2-hydroxy-2-methyl-1-propanone, 2,2-dimethoxy-2-phenylacetophenone (DMPA) or riboflavin.

The ocular composition of the invention may further comprise a co-initiator. Optionally, the photoinitiator is riboflavin and the co-initiator is L-arginine.

The ocular composition of the invention may be a nanoparticle or a microparticle ocular implant. Optionally, the nanoparticle ocular implant is less than about 1,000 nm. Optionally, the microparticle ocular implant is less than about 1,000 μm.

According to a second aspect of the invention, there is provided a method of making the ocular composition of the first aspect of the invention, comprising the steps of:
i) mixing the therapeutic agent, the photopolymerizable composition, the biodegradable polymer and the photoinitiator, in any order of addition, to form mixture i);
ii) administering the mixture i) to an ocular area of a subject; and
iii) irradiating the mixture i) with light at a wavelength of between 230 to 550 nm, between 300 to 525 nm, or between 350 to 490 nm for between 1 second and 60 minutes to form the ocular composition.

Optionally, the irradiating step is with light at a wavelength of 365 nm or 475 nm for 1 second, 2 minutes, 5 minutes, 10 minutes, 20 minutes, or 30 minutes.

According to a third aspect of the invention, there is provided a method of making the ocular composition of the first aspect of the invention, the method comprising the steps of:
i) mixing the therapeutic agent, the photopolymerizable composition, the biodegradable polymer and the photoinitiator, in any order of addition, to form mixture i);
ii) irradiating the mixture i) with light at a wavelength of between 230 to 550 nm, between 300 to 525 nm, or between 350 to 490 nm for between 1 second and 60 minutes to form the ocular composition; and
iii) administering the composition ii) to an ocular area of a subject.

Optionally, the irradiating step is with light at a wavelength of 365 nm or 475 nm for 1 second, 2 minutes, 5 minutes, 10 minutes, 20 minutes, or 30 minutes.

According to a fourth aspect of the invention, there is provided a method of making the nanoparticle or microparticle ocular implant, comprising the steps of:
i) mixing the therapeutic agent, the photopolymerizable composition, the biodegradable polymer and the photoinitiator, in any order of addition, to form mixture i);
ii) adding the mixture i) to an aqueous medium to form mixture ii);
iii) sonicating the mixture ii); and
   irradiating the mixture ii) with light at a wavelength of between 230 to 550 nm, between 300 to 525 nm, or between 350 350 to 490 nm for between 1 second and 60 minutes to form the nanoparticles or microparticles.

### BRIEF DESCRIPTION OF THE DRAWINGS

FIG. 1: Digital images of blank (without drug) ISPcl showing (A) in situ implant formation in PBS and (B) degradation of implant after 160 days. The ISPcl were composed of 30% w/w PLGA 75/25, 0.1% w/w Igracure® 2959 and 69.9% w/w of PEGDA 700. The gels were in situ crosslinked in PBS using UV light (at 365 nm, 3.14 mW/cm²) for 5 min. Mean±S.D, n=3.

FIG. 2: In vitro release of dexamethasone (DEX) from ISPcl containing 30% w/w PLGA 75/25, 0.5% w/w DEX, and 0.1% w/w of Igracure® 2959 and 69.4% w/w of PEGDA 700. The gels were in situ crosslinked, using UV light at 365 nm, for 5, 10, 20 and 30 min, respectively. Mean±S.D, n=3

FIG. 3: In vitro release of bovine serum albumin (BSA) from ISPcl containing 30% w/w PLGA 75/25, 0.5% w/w BSA, and 0.1% w/w of Igracure® 2959 and 69.4% w/w of PEGDA 700. The gels were in situ crosslinked, using UV light at 365 nm 5 min. Mean±S.D, n=3

FIG. 4: In vitro release of bevacizumab (Avastin) from ISPcl containing 30% w/w PLGA 75/25, 1.5% w/w bevacizumab, 0.1% w/w Irgacure® and 68.4% w/w PEGDA 700 and 30% w/w PLGA 75/25, 1.5% w/w bevacizumab, 0.05% w/w Irgacure® and 68.45% w/w PEGDA 700. In situ crosslinked using UV light at 365 nm for either 30 sec or 2.5 min. Mean±S.D, n=3

FIG. 5: In vitro release of ovalbumin (OVA) from ISPcl containing 30% w/w PLGA 75/25, 2.5% w/w OVA and 0.1% w/w Irgacure® 2959 and 67.4% w/w of PEGDA 700. In situ crosslinked for either 30 sec, 1 min and 2.5 min. Mean±S.D, n=3.

FIG. 6: Work of Syringeability (WoS) of 30% w/w PLGA50/50 and 30% w/w PLGA75/25 and 0.1% w/w Irgacure® 2959 in formulations containing different molecular weights of 69.9% w/w of PEGDAs (258, 575 and 700). The WoS was calculated from the resultant of force-distance plots Mean±S.D, n=5.

FIG. 7: Percentage cell viability of human retinal pigment epithelial cell line (ARPE-19) following exposure of the release media, at different intervals, of the ISPcl that were composed of 30% w/w PLGA 75/25, 0.1% w/w of Igracure® 2959 and 69.9% w/w PEGDA 700 and UV crosslinked for 5 min. Mean±SD, n=3.

FIG. 8: (A) Scanning electron microscope image of PPcl implant (scale bar is 50 μm). (B) Digital image of a PPcl adjacent to a ruler.

FIG. 9: In vitro release of FITC-Dextran 150 kDa and BSA from PPcl containing 30% w/w of PLGA 75/25, 0.5% w/w FITC-Dextran 150 kDa or BSA and 0.1% w/w of Igracure® 2959 and 69.4% w/w PEGDA 700. These PPcl were cross-linked using UV curing system at wavelength 365 nm for 5 runs at a lamp intensity of 100%.

FIG. 10: In vitro release of TA from PPcl containing 30% w/w of PLGA 75/25, 2.5% w/w TA, and 0.1% w/w of Igracure® 2959 and 67.4% w/w PEGDA 700. These PPcl were cross-linked using UV curing system at wavelength 365 nm for 5 runs, at a lamp intensity (LI) of 50% and 100%.

FIG. 11: In vitro release of TA from PPcl containing 2.5 or 5% w/w of PLGA 75/25, 2.5% w/w TA, 0.1% w/w of Igracure® 2959 in 94.9% w/w or 92.4% w/w of PEGDA 700, respectively. These PPcl were cross-linked using UV curing system at wavelength 365 nm for 5 runs, at a lamp intensity (LI) of 25%.

FIG. 12: In vitro release of TA from PPcl containing 2.5% w/w of PLGA 75/25, 2.5% w/w TA, 0.1% w/w of Igracure® 2959 and without pore forming agent in 94.9% w/w PEGDA 700 or with 2% w/w of pore forming agent (i.e. MgCO₃) in 92.9% w/w PEGDA 700. These PPcl were cross-linked using UV curing system at wavelength 365 nm for 10 runs, at a lamp intensity (LI) of 25%.

FIG. 13: In vitro release of TA from PPcl containing 2.5% w/w of PLGA 75/25, 2.5% w/w TA, 0.1% w/w of Igracure® 2959 in 94.9% w/w of PEGDA 700 or PEGDA 6000. These PPcl were cross-linked using UV curing system at wavelength 365 nm for 10 runs, at a lamp intensity (LI) of 25%.

FIG. 14: In vivo implant formation of ISPcl in rat eye following intravitreal injection (A) Digital image of rat following administration of fluorescein sodium loaded ISPcl implant (2 μL), the inset shows close-up image of the implant in the eye, (B) shows fundus image of control eye without any implant, (C-E) fundus images showing the time course of fluorescein sodium release from ISPcl implant, taken on (C) day 1, (D) day 6 and (E) day 18. Implants indicate slow and continuous release of fluorescein sodium and degradation over the time.

### DETAILED DESCRIPTION

### Photopolymerizable Compositions

The photopolymerizable polymers of the present invention can be used in any of the compositions and implants of the invention in combination with any of the other biodegradable polymers, therapeutic agents, photoinitiators, solvents, co-solvents, pore forming agents, and co-initiators described herein or known in the common general knowledge.

In one embodiment, the photopolymerizable compositions of the invention can be biodegradable. As used herein, “biodegradable” is the chemical degradation by biological means. In some embodiments, the biodegradation is about 100%, about 98%, about 90%, about 85%, about 80%, about 60%, about 50%, or about 45% degradation of one or more of the compositions, monomers, oligomers, fragments, polymers, photoinitiators, solvents, co-solvents, or co-initiators. In some embodiments the biodegradation takes place over about 1 minute, about 10 minutes, about 20 minutes, about 2 hours, about 6 hours, about 12 hours, about 24 hours, about 2 days, about 5 days, about 1 week, about 1 month, about 2 months, about 5 months, about 6 months, about 8 months or about 12 months. In some embodiments the biodegradation takes place between about 1 month and about 12 months, between about 6 months and about 12 months, or between about 8 months and about 12 months.

As used herein, the term “photopolymerizable composition” is a composition which can form a crosslinked polymer network upon exposure to light, in particular UV light. As used herein, photopolymerizable compositions include photopolymerizable monomers and oligomers (such as, dimers, trimers, and tetramers). The terms “oligomers” and “fragments” can be used interchangeably to mean between two and twenty monomers, optionally between two and ten monomers, further optionally between two and five monomers or between two and four monomers. A “photopolymerizable monomer” is a single unit of a photopolymerizable polymer that can bind chemically to other monomers to form a polymer.

Photopolymerizable compositions of the present invention can be crosslinked with UV radiation to form photopolymerized polymers of the present invention.

In one embodiment the photopolymerizable compositions of the present invention are fragments or monomers consisting of polyalkylene glycol diacrylate, polyalkylene glycol dimethacrylate and mixtures, copolymers, and block copolymers thereof.

In one embodiment, the photopolymerizable compositions are polyalkylene glycol diacrylate fragments or monomers incorporating diacrylate end units selected from the group comprising polyether fragments or monomers, polyester fragments or monomers, polycarbonate fragments or monomers or mixtures, copolymers, or block copolymers thereof. In one embodiment, the photopolymerizable composition is a monomers incorporating diacrylate end units, such as 4-arm or 8-arm PEG acrylate.

In another embodiment, the photopolymerizable composition is polyethylene glycol diacrylate, diethylene glycol diacrylate, polyethylene glycol dimethacrylate, diethylene glycol dimethacrylate, polypropylene glycol diacrylate, dipropylene glycol diacrylate, dipropylene glycol dimethacrylate, and polypropylene glycol dimethacrylate or mixtures, copolymers, or block copolymers thereof.

In another embodiment, the photopolymerizable composition is polyethylene glycol diacrylate or polyethylene glycol dimethacrylate.

In yet another embodiment, the photopolymerizable composition is polyethylene glycol diacrylate.

The molecular weight of the photopolymerizable compositions of the present invention is typically between about 100 and about 300,000 Da, between about 200 to about 100,000 Da, between about 200 to 50,000 Da, between about 200 to about 20,000 Da, between about 200 to about 10,000 Da, between about 200 and about 8,000 Da, between about 200 and about 5,000 Da, or between about 200 and about 1,000 Da.

It has been found, for the compositions and implants of the present invention, that an increase in molecular weight of the photopolymerizable compositions results in an increase in drug release rate. Without wishing to be bound by theory, it is believed that photopolymerizable compositions with lower molecular weights have higher crosslinking density and therefore slower drug release rates.

The photopolymerizable compositions of the present invention typically have viscocities between about 0.1 to about 7 dL/g, between about 0.2 to about 5 dL/g, or between about 0.5 to 2 dL/g.

In another embodiment, the photopolymerizable compositions of the present invention are polymerized by irradiating the composition with light at a wavelength of between about 230 to about 550 nm, between about 300 to about 525 nm, or between about 350 to about 490 nm for between about 1 second and about 60 minutes, between about 30 seconds and about 30 minutes, between about 2.5 minutes and about 20 minutes, between about 5 minutes and about 10 minutes. In one embodiment, the crosslinking is for about 30 seconds, about 1, about 2.5, about 5, about 10, about 20 or about 30 minutes.

### Biodegradable Polymers

The biodegradable polymers of the present invention can be used in any of the compositions and implants of the invention in combination with any of the other photopolymerizable compositions, therapeutic agents, photoinitiators, solvents, co-solvents, pore forming agents, and co-initiators described herein or known in the common general knowledge.

The biodegradable polymers of the present invention are biodegradable but not photopolymerizable.

In one embodiment of the present invention, the biodegradable polymers are aliphatic polyester-based polyurethanes, polylactides, polycaprolactones, polyorthoesters or mixtures, copolymers, or block copolymers thereof. In another embodiment of the present invention the biodegradable polymer, is chitosan, poly(propylene fumarate), lactide/glycolide copolymer (PLGA), polylactic acid (PLA), polyglycolic acid (PGA), polycaprolactone (PCL), lactide/caprolactone copolymer (PLC), poly (L-lactide) (PLLA), natural biodegradable polymers, such as, collagen and the like, or mixtures, copolymers, or block copolymers thereof. In another embodiment, the biodegradable polymer is selected from the group lactide/glycolide co-polymer (PLGA), polylactic acid (PLA), polyglycolic acid (PGA), polycaprolactone (PCL), lactide/caprolactone copolymer (PLC), poly (L-lactide) (PLLA) or mixtures, copolymers, or block copolymers thereof.

In a particular embodiment, the biodegradable polymer is PLGA. In one embodiment, the molar ratio of lactic acid to glycolic acid in the PLGA is 90% lactic acid to 10% glycolic acid, 85% lactic acid to 15% glycolic acid, 75% lactic acid to 25% glycolic acid, 65% lactic acid to 35% glycolic acid, 50% lactic acid to 50% glycolic acid, 35% lactic acid to 65% glycolic acid, 25% lactic acid to 75% glycolic acid, 15% lactic acid to 85% glycolic acid, and 10% lactic acid to 90% glycolic acid.

In another particular embodiment, the biodegradable polymer is PCL, PLC, PLLA, or mixtures, copolymers, or block copolymers thereof.

### Compositions and Implants

In one embodiment, the compositions of the invention comprise combinations of photopolymerizable compositions, and biodegradable polymers, as described above, in combination with a photoinitiator and a therapeutic agent, which can be delivered to the eye to achieve controlled drug delivery to treat a range of eye diseases. The compositions of the invention include:
i) compositions which can be injected into the eye followed by application of short-term UV light to induce in situ photocrosslinking, resulting in implant formation, termed as in situ photocrosslinked implants (ISPcl); and
ii) compositions which can be photocrosslinked prior to application in the eye to form an implant of desired shape and size (e.g. film, rod or nano/microparticles) that can be administered intraocularly to provide desired period of drug delivery, termed as performed photocrosslinked implants (PPcl).

Alternatively, the compositions of the invention can be used to coat ocular devices, including both in situ and pre-formed ocular devices.

The implants of the present invention can be any desired shape and size, such as but not limited to, for example rectangular, square, cylindrical, circular, oval, films, dumbbell, rods, beads, etc., as, for example, macro, micro or nanoparticles.

In one embodiment of the present invention, the ocular implant is a implant, which is less than about 10 mm, less than about 5 mm. In one embodiment, the implant is a rectangular implant of dimensions 10×5×0.5 mm.

In one embodiment of the present invention, the ocular implant is a nanoparticle or a microparticle.

In one embodiment, the nanoparticle ocular implant is less than about 1,000 nm, less than about 900 nm, less than about 750 nm, less than about 500 nm, or less than about 100 nm.

In one embodiment, the microparticle ocular implant is less than about 1,000 μm, less than about 900 μm, less than about 750 μm, less than about 500 μm, or less than about 25 μm.

### In Situ Photocrosslinked Implants (ISPcl)

In situ photocrosslinked implants (ISPcl), of the present invention are those that form and take up their final localised structure once they are inserted into the body. The ability of these implants to fill irregular defects is an advantage of ISPcls. The ISPcls of the present invention also have additional advantages, which include, site-specific action due to relatively easy and less invasive application, localized delivery to specific tissues, prolonged delivery times, reduction in side effects linked with systemic delivery and also superior patient comfort and compliance. Additional advantages of the ISPcls of the present invention include, not requiring extreme pH conditions or elevated temperatures during processing, which could cause issue when working with temperature or pH labile drugs (e.g. proteins, peptides or genetic material). Furthermore, rapid crosslinking at physiological temperatures can swiftly entrap drug molecules and can result in an ISPcl that possesses the exact required dimensions for controlled drug release. Photocrosslinking is also beneficial in comparison to spontaneous crosslinking (e.g. enzymatic, self-assembled, Michael addition) as the initiation of the process is only triggered when exposed to a light source, therefore premature gelation is not an issue resulting in excellent control of material formation. Furthermore, short-term application of UV light will not cause any safety issues as it is considered safe for ocular applications, as UV light is clinically used for corneal crosslinking. Importantly, administration by this method allows the injection of a relatively low viscosity material into the body, which then solidifies to form a semi-solid depot that controls the drug delivery to provide short or long-term therapeutic action.

In one embodiment, the ISPcls of the present invention are formed by injection of a composition of the invention into a subject in need thereof and subsequent crosslinking using external source of UV light that results in formation of a solid implant which controls drug release for desired period of time.

For ISPcls of the invention the molecular weight of the photopolymerizable composition is typically between about 100 and about 6,000 Da, between about 200 and about 3,000 Da, or between about 200 and 1,000 Da.

### Preformed Photocrosslinked Implants (PPcl)

In one embodiment, the present invention is a preformed photocrosslinked implant (PPcl). These PPcls can be inserted in the eye (e.g. in the fornix, subconjunctively, intracameral, intrastromal/intracorneal, transsclerally/periocular, intrasclerally or intravitreally) to treat the front of the eye or back of the eye diseases. These implants can be fabricated in a variety of shapes (e.g. rods, films, cylindrical or circular) and sizes, including in the form of micro or nanoparticles.

The PPcls of the present invention have the advantage of high crosslink density and/or a tight polymer network structure which can be configured to control drug release and/or eliminate any burst release.

The PPcls of the present invention can be fabricated to have a single and/or multiple layers, which will enable loading of more than one drug or the same drug with different release profiles or rates.

Furthermore, the rate of degradation of the implants can be slower for PPcls when compared to ISPcls of the invention and can be altered to treat specific diseases or disorders.

For the PPcls of the invention the molecular weight of the photopolymerizable polymers is typically between about 100 and about 300,000 Da, between about 200 to 100,000 Da, between about 200 to 50,000 Da, between about 200 to 20,000 Da, or between about 200 to about 10,000 Da.

In one embodiment, the biodegradable polymer is essentially contained within a matrix of the photopolymerizable composition. In one embodiment, the biodegradable polymer is essentially contained within a matrix of the photopolymerizable composition that forms a gel upon mixing. In one embodiment the photopolymerizable polymer is crosslinked in presence of a photoinitiator and the biodegradable polymer and therapeutic agent(s). In one embodiment, the biodegradable polymer is essentially trapped within the crosslinked photopolymerizable polymer matrix, and the therapeutic agent(s) are either dispersed or dissolved between the two phases (i.e., photopolymerizable and/or biodegradable polymer). In one embodiment, the biodegradable polymer is hydrophobic in nature and the photopolymerizable polymer is hydrophilic in nature. In one embodiment, the degree of crosslinking of the composite implant will govern the rate and extent of release of the therapeutic agent(s).

In one embodiment, the present invention is an ocular composition comprising:
i) 99 to 60% (w/w) of a photopolymerizable composition selected from the group of fragments or monomers consisting of polyalkylene glycol diacrylate and polyalkylene glycol dimethacrylate, wherein the photopolymerizable composition has a molecular weight in the range of 200 to 20,000 Dalton;
ii) a biodegradable polymer selected from the group consisting of aliphatic polyester-based polyurethanes, polyglycolide, polylactides, polycaprolactones, polyorthoesters and mixtures, copolymers, and block copolymers thereof;
iii) a photoinitiator; and
iv) a therapeutic agent.

In one embodiment, the photopolymerizable composition is 96.9% (w/w) and the biodegradable polymer is 2.5% (w/w), the photopolymerizable composition is 94.1% (w/w) and the biodegradable polymer is 5% (w/w), the photopolymerizable composition is 69.4% (w/w) and the biodegradable polymer is 30% (w/w).

In another embodiment, the present invention is an ocular composition wherein i) and ii) are:
i) 79.5 to 59.5% (w/w) polyethylene glycol diacrylate or polyethylenene glycol dimethacrylate; and
ii) 20 to 40% (w/w) PLGA, wherein the molar ratio of lactic acid to glycolic acid in the PLGA is 90% lactic acid to 10% glycolic acid, 85% lactic acid to 15% glycolic acid, 75% lactic acid to 25% glycolic acid, or 50% lactic acid to 50% glycolic acid.

In another embodiment, the present invention is an ocular composition wherein i) and ii) are:
i) 79.5% (w/w) polyethylene glycol diacrylate or polyethylene glycol dimethacrylate; and
ii) 30% (w/w) PLGA wherein the molar ratio of lactic acid to glycolic acid in the PLGA is 90% lactic acid to 10% glycolic acid, 85% lactic acid to 15% glycolic acid, 75% lactic acid to 25% glycolic acid, or 50% lactic acid to 50% glycolic acid.

In another embodiment, the present invention is an ocular composition wherein i) and ii) are:
i) 95.5 to 84.5% (w/w) polyalkylene glycol diacrylate or polyalkylene glycol dimethacrylate; and
ii) 4 to 15% (w/w) PCL.

In another embodiment, the present invention is an ocular composition wherein i) and ii) are:
i) 69.5 to 94.5% (w/w) polyalkylene glycol diacrylate or polyalkylene glycol dimethacrylate; and
ii) 20 to 5% (w/w) PLLA.

In yet another embodiment, the present invention is an ocular composition wherein i) and ii) are:
i) 95.5 to 84.5% (w/w) polyalkylene glycol diacrylate or polyalkylene glycol dimethacrylate; and
ii) 4 to 15% (w/w) PLC in which lactic acid to caprolactone is in the range of 90% lactic acid to 10% caprolactone, 80% lactic acid to 20% caprolactone, 70% lactic acid to 30% caprolactone, 60% lactic acid to 40% caprolactone, or 50% lactic acid to 50% caprolactone.

In one embodiment, the present invention is an ocular composition wherein i) is 95.5 to 84.5% (w/w) polyalkylene glycol diacrylate or polyalkylene glycol dimethacrylate; and wherein ii) is 4 to 15% (w/w) PCL.

In one embodiment, the % of the biodegradable polymer is 30% w/w, 5% w/w. 2.5% w/w, between 4-10% w/w, or between 5-18% w/w.

In one embodiment, i) and ii) of the compositions of the present invention are PEGDA and PLGA.

### PLGA

PLGA is prepared by polymerisation of lactic acid and glycolic acid monomers. The glass transition temperatures (T₉) of PLGA copolymers are above physiological temperatures of 37° C., which imparts a moderately rigid chain configuration and therefore the mechanical strength at ambient temperatures. The use of PLGA in different lactide (LA) to glycolide (GA) ratio and molecular weight allows different drug release profiles. An increase in GA content will result in an increased water uptake of PLGA and therefore more rapid degradation. The degradation of PLGA with LA/GA 50/50 is typically between about 1 and about 3 months.

### PEGDA

PEGDA is a synthetic polymer, available in different M_{w}. PEGDA is extremely amenable to mechanical, structural and chemical alteration and so resulting in hydrogels with variable properties in terms of drug delivery and other biomedical applications. PEGDA is formed through the functionalization of the end of each PEG molecule with an acrylate group. PEGDA is also non-toxic, eliciting only a minimal immunogenic response. PEGDA has double-bond containing acrylate end groups which show rapid polymerisation when exposed to light in the presence of an appropriate initiator to produce a hydrogel network.

In one embodiment, the present invention is a PLGA/PEGDA PPcl.

In one embodiment, the present invention is a PLGA/PEGDA ISPcl.

### Copolymers

All of the copolymers and block copolymers described herein can be used in any of the compositions and implants of the invention in combination with any of the other photopolymerizable compositions, biodegradable polymers, therapeutic agents, photoinitiators, solvents, co-solvents, pore forming agents, and co-initiators described herein.

As used herein “copolymer” is a mixture of two or more different types of monomer units. As used herein “block copolymer” is a mixture of two or more homopolymer subunits.

In one embodiment, block or copolymers with PGA, PCL, PLA, PLGA that would include any other polymeric component of the polymer e.g. PEG and PEO, for example, PLGA-PEO, PCL-PEO and PEG-PLGA, PEG-PCL block copolymers, which include, for example, PEO-PLGA-PEO, PLGA-PEG, PLGA-PEO, and PLGA-PEO-PLGA.

### Solvents

All of the solvents described herein can be used in the preparation of any of the compositions and implants of the invention in combination with any of the other photopolymerizable compositions, biodegradable polymers, therapeutic agents, photoinitiators, pore forming agents, and co-initiators described herein.

In one embodiment, the co-solvents used in the present invention can be selected from dichloromethane, tetrahydrofuran, ethyl acetate, acetone, dimethylformamide, acetonitrile, acetic acid, methanol, ethanol, isopropanol, glycofurol or butanol.

In one embodiment, the solvents used in the present invention are dimethyl sulfoxide, decylmethyl sulfoxide, 2-pyrrolidone, N-vinyl-2-pyrrolidone, 1-methyl-2-pyrrolidone, N-Methyl-2-pyrrolidone, N-ethyl-pyrrolidone, glycerol formal, glycerol, polyethylene glycol, propylene glycol, benzyl alcohol, benzyl benzoate, ethyl benzoate, triacetin, triethyl citrate, dimethylformamide, dimethylacetamide or tetrahydrofuran.

In another embodiment, the solvent is dimethyl sulfoxide, decylmethyl sulfoxide, 2-pyrrolidone, 1-methyl-2-pyrrolidone, N-Methyl-2-pyrrolidone, or glycerol formal.

In one embodiment, a solvent is used when the biodegradable polymer is PCL, PLC and/or PLLA. In one embodiment the solvent is N-Methyl-2-pyrrolidone and N-Vinyl-2-pyrrolidine when the biodegradable polymer is PCL, PLC and/or PLLA. In another embodiment, a solvent is used when the photomolymerizable composition is PEGDA.

### Pore Forming Agents

In one embodiment, a suitable pore forming agent may be selected in view of the specific therapeutic agent and composition of the implant, and the desired elution profile or release rate. The pore forming agent may be a naturally occurring agent or polymer or a synthetic agent or polymer.

In another embodiment, implant porosity can be adjusted by preparing implants in the presence of dispersed water-soluble porosigens, which can be removed later by washing with water to leave an interconnected meshwork (i.e., porous hydrogels). The pore size of hydrogels prepared by the porosigen technique depends on the size of the porosigens.

All of the pore forming agents described herein can be used in any of the implants and compositions of the invention in combination with any of the other photopolymerizable compositions, biodegradable polymers, therapeutic agents, photoinitiators, solvents, co-solvents, and co-initiators described herein.

In one embodiment, the compositions of the invention further comprise a pore-forming agent.

In one embodiment, the pore-forming agent is polyethylene glycol, lactose, maltose, glucose, mannitol, gelatin, sodium chloride, magnesium carbonate, magnesium hydroxide, potassium chloride, sodium bicarbonate, ammonium bicarbonate, potassium bicarbonate, chitosan, polyvinylpyrrolidone, polyvinyl alcohol, agarose or sucrose.

### Photoinitiators

In one embodiment, the compositions of the invention further comprise a photoinitiator.

The photoinitiators described herein can be used in any of the compositions and implants of the present invention in combination with any of the other photopolymerizable compositions, biodegradable polymers, therapeutic agents, photoinitiators, solvents, co-solvents, and co-initiators described herein.

In certain embodiments, the photoinitiator is designed to work using light from about 200 to about 550 nm. In some embodiments, a photoinitiator is designed to work using UV light from about 200 to about 400 nm.

In certain embodiments, the light source may allow variation of the wavelength of light and/or the intensity of the light. Light sources useful in the present invention include, but are not limited to, lamps, fiber optics devices, etc.

In one embodiment, the photoinitiator is a ketone (e.g., RCOR′). In one embodiment, the compound is an azo compound (e.g., compounds with a —N═N— group). In one embodiment, the photoinitiator is an acylphosphineoxide. In one embodiment, the photoinitiator is a sulfur-containing compound. In one embodiment, the initiator is a quinone. In certain embodiments, a combination of photoinitiators is used.

In one embodiment, the photoinitiator is a hydroxyketone photoinitiator, an amino ketone photoinitiator, a hydroxy ketone/benzophenone photoinitiator, a benzyldimethyl ketal photoinitiator, a phenylglyoxylate photoinitiator, an acyl phosphine oxide photoinitiator, an acyl phosphine oxide/alpha hydroxy ketone photoinitiator, a benzophenone photoinitiator, a ribityl isoalloxazine photoinitiator, or a phenylglyoxylate photoinitiator or any combination thereof.

In one embodiment the photoinitiator is 1-[4-(2-hydroxyethoxy)-phenyl]-2-hydroxy-2-methyl-1-propanone, 2,2-dimethoxy-2-phenylacetophenone (DMPA) or riboflavin.

In one embodiment, the compositions of the present invention further comprise a co-initiator. In one embodiment, the co-initiator is eosin Y, triethanolamine, camphorquinone, 1-vinyl-2 pyrrolidinone (NVP), eosin, dimethylaminobenzoate (DMAB), Irgacure® 907 (Ciba Geigy), Irgacure® 651 (Ciba Geigy), Darocur 2959 (Ciba Geigy), or ethyl-4-N,N-dimethylaminobenzoate (4EDMAB).

In one embodiment, the photoinitiator is riboflavin and the co-initiator is L-arginine.

### Process

The compositions and implants of the present invention can be made by any methods know in the art as well as the methods described herein. The methods described herein are applicable to all compositions and implants of the invention.

In one embodiment, polymer M_{w}, type and copolymer ratio, drug type and loading, implant size, time and extent of UV crosslinking and/or amount and type/concentration of photoinitiator and/or pore forming agent (porogen) and/or solvent/co-solvent can be altered to control the rate and extent of drug release. The alteration of these factors provides compositions of the invention that can be easily tailored to produce desired period of drug release to address specific clinical/patient needs in treating various ocular diseases.

In one embodiment, the present invention is a method of making the ocular composition of the invention, comprising the steps of:
i) mixing a therapeutic agent with a photopolymerizable composition, a biodegradable polymer, and a photoinitiator, in any order of addition;
ii) administering the mixture i) to an ocular area of a subject; and
iii) irradiating the mixture i) with light at a wavelength of between about 230 to about 550 nm, between about 300 to about 525 nm, or between about 350 to about 490 nm for between about 1 second and about 60 minutes to form the ocular composition.

In another embodiment the present invention is a method of making the ocular composition of the present invention, comprising the steps of:
i) mixing a therapeutic agent with a photopolymerizable composition;
ii) adding a biodegradable polymer to the mixture i)
iii) adding a photoinitiator to the mixture ii);
iv) administering a mixture iii) to an ocular area of a subject; and
v) irradiating the mixture iii) with light at a wavelength of between about 230 to about 550 nm, between about 300 to about 525 nm, or between about 350 to about 490 nm for between about 1 second and about 60 minutes to form the ocular composition.

In the above embodiments, the irradiating step is with light at a wavelength of about 365 nm or about 475 nm for about 1 second, about 2 minutes, about 5 minutes, about 10 minutes, about 20 minutes, or about 30 minutes.

In another embodiment, the present invention is a method of making the ocular compositions of the present invention, the method comprising the steps of:
i) mixing a therapeutic agent with a photopolymerizable composition, a biodegradable polymer and a photoinitiator, in any order of addition;
ii) irradiating the mixture i) with light at a wavelength of between about 230 to about 550 nm, between about 300 to about 525 nm, or between about 350 to about 490 nm for between about 1 second and about 60 minutes to form the ocular composition; and
iii) administering the composition ii) to an ocular area of a subject.

In another embodiment, the present invention is a method of making the ocular composition of the present invention, the method comprising the steps of:
i) mixing a therapeutic agent with a photopolymerizable composition;
ii) adding a biodegradable polymer to the mixture i)
iii) adding a photoinitiator to the mixture ii);
iv) irradiating the mixture iii) with light at a wavelength of between about 230 to about 550 nm, between about 300 to about 525 nm, or between about 350 to about 490 nm for between about 1 second and about 60 minutes to form the ocular composition; and
v) administering the composition iv) to an ocular area of a subject.

In the above embodiments, the irradiating step is with light at a wavelength of about 365 nm or about 475 nm for about 1 second, about 2 minutes, about 5 minutes, about 10 minutes, about 20 minutes, or about 30 minutes.

In another embodiment, the present invention is a method of making the nanoparticle or microparticle ocular implant, comprising the steps of:
i) mixing the therapeutic agent, the photopolymerizable composition, the biodegradable polymer and the photoinitiator, in any order of addition, to form mixture i);
ii) adding the mixture i) to an aqueous medium to form mixture ii);
iii) sonicating the mixture ii); and
   irradiating the mixture ii) with light at a wavelength of between 230 to 550 nm, between 300 to 525 nm, or between 350 to 490 nm for between 1 second and 60 minutes to form the nanoparticles or microparticles

In another embodiment, the present invention is a method of making the nanoparticle or microparticle ocular implants of the present invention, comprising the steps of:
i) mixing a therapeutic agent with a photopolymerizable composition;
ii) adding a biodegradable polymer to the mixture i)
iii) adding a photoinitiator to the mixture ii);
iv) adding the mixture iii) to a aqueous medium;
v) sonicating the mixture iv); and
vi) irradiating the mixture v) with light at a wavelength of between about 230 to about 550 nm, between about 300 to about 525 nm, or between about 350 to about 490 nm for between about 1 second and about 60 minutes to form the nanoparticles or microparticles.

In one embodiment the aqueous medium is a combination of water and phosphate buffered saline (PBS).

In one embodiment, step iv) is carried out in a step-by-step manner to emulsify the mixture. As used herein step-by-step means that the mixture is not added all at once, but rather it is added in stages with breaks of between the additions.

In another embodiment, i) the therapeutic agent is mixed with a portion of photopolymerizable composition and ii) another portion of photopolymerizable composition is mixed with biodegradable polymer, then iii) the two portions are mixed together. The mixture iii) is added to a aqueous medium, and then sonicated. Finally the mixture is irradiated with light at a wavelength of between about 230 to about 550 nm, between about 300 to about 525 nm, or between about 350 to about 490 nm for between about 1 second and about 60 minutes to form the nanoparticles or microparticles.

In yet another embodiment, irradiation can be applied during sonication i.e. sonicating the mixture under UV light, in other words, the aqueous medium will be under UV light (at defined wavelength) and sonication, followed by step-by-step addition of the mixture.

In another embodiment, the sonication time, gel composition, phase ratio (of the gel vs aqueous medium), and rate of adding the gel mixture into aqueous medium are adjusted to form a nanoparticle or microparticle.

In the compositions of the present invention, varying the UV crosslinking time can control the rate of and duration of drug release. In some embodiments, an increase in UV crosslinking times causes a decrease in drug release. Additionally, varying the concentration of the photoinitiator can control the rate and duration of drug release. Furthermore, varying both the UV crosslinking time and the concentration of photoinitiator can control the rate and duration of drug release. In one embodiment, decreasing the concentration of the biodegradable polymer (such as PLGA) increases the drug release rate. In one embodiment, adding a pore-forming agent (e.g. MgCO₃), increases the drug release rate. In one embodiment, higher UV crosslinking time and higher concentration of photoinitiator can sustain the drug release for longer periods of time. In one embodiment, the drug release can be sustained for a period of greater than about 1 day, about 2 days, about 1 week, about 1 month, about 2 months, about 3 months, or about 6 months.

In one embodiment, the duration of drug release in the ISPcls of the present invention can be considerably extended, for example, providing controlled drug release for a period of greater than 200 days (>6 months). This duration can be varied by varying the degree of crosslinking.

In some embodiments, the slow degradation rate of the ISPcls of the present invention provide protection of the sensitive molecules such as peptides and proteins. It has been shown below, that the ISPcls of the present invention are stable and avoid protein degradation and maintain protein activity.

In some embodiments, burst release can be eliminated or controlled by varying the UV crosslinking time.

In one embodiment, the present invention is a PPcl with no burst release. In one embodiment, the present invention is a PPcl with high crosslinking density that significantly slows drug diffusion.

### Methods of Use

Any of the implants and compositions described herein are suitable for use in any of the methods of the invention described herein.

In one embodiment, the present invention is a method of treating a disease or disorder of the eye in a subject in need thereof, comprising administering a composition or implant of the present invention to an ocular area of the subject.

In one embodiment, the present invention is a composition or implant of the present invention for use in treating a disease or disorder of the eye in a subject in need thereof.

As used herein, an “ocular area” is an area inside, outside or adjacent to the eye of the subject. In one embodiment, the ocular area is the sclera (intrascleral), outside the sclera (transscleral), the vitreous body, the choroid, the cornea, the stroma, intracameral, the aqueous humor, the lens, the fornix, or the optic nerve.

In one embodiment, the compositions and implants can be administered by injection, including, intravitreal, subconjunctival, peribulbar, subtenon or retrobulbar injections and cornea.

In some embodiments, the implants are administered via a surgical procedure. In some embodiment, the implants are secured in place, after surgical implantation, via an adhesive or sutures.

The term “subject” refers to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In one embodiment, the subject is a non-human animal such as a farm animal (e.g., a horse, cow, pig or sheep), or a pet (e.g., a dog, cat, guinea pig or rabbit). In another embodiment, the subject is a “human”.

As used herein, the terms “treat”, “treatment” and “treating” refer to therapeutic treatments includes the reduction or amelioration of the progression, severity and/or duration of a disease, disorder or condition, or the amelioration of one or more symptoms (specifically, one or more discernible symptoms) of a disease, disorder or condition, resulting from the administration of the compositions or implant of the invention. In specific embodiments, the therapeutic treatment includes the amelioration of at least one measurable physical parameter of a disease, disorder or condition. In other embodiments the therapeutic treatment includes the inhibition of the progression of a condition, either physically by, e.g., stabilization of a discernible symptom, physiologically by, e.g., stabilization of a physical parameter, or both. In other embodiments the therapeutic treatment includes the reduction or stabilization of a disease, disorder or condition.

Exemplary therapeutic agents include, but are not limited to, polypeptides, nucleic acids, such as DNA, RNA, and siRNA, growth factors, steroid agents, antibody therapies, antimicrobial agents, antibiotics, antiretroviral drugs, anti-inflammatory compounds, antitumor agents, anti-angiogeneic agents, and chemotherapeutic agents.

In one embodiment, the therapeutic agent of the present invention includes, but is not limited to, ketorolac, naphazoline, lidocaine, bevacizumab, aflibercept, pegaptanib, brimonidine, dorzolamide, azithromycin, rapamycin, bepotastine besilate, diclofenac, besifloxacin, cysteamine hydrochloride, fluocinolone acetonide, difluprednate, aflibercept, tasimelteon, ocriplasmin, enoxaparin sodium, ranibizumab, latanoprost, timolol, bimatoprost, pegaptanib, ofloxacin, cephazolin, phenylephrine, dexamethasone, triamcinolone acetonide, levofloxacin, cyclophosphamide, melphalan cyclosporine, methotrexate, azathioprine ketorolac, travoprost, verteporfin, tafluprost, ketotifen fumarate, foscarnet, amphotericin B, fluconazole, voriconazole, ganciclovir, acyclovir, gatifloxacin, vitamin (vitamin A, vitamin C, and vitamin E), zinc, copper, lutein, zeaxanthin or combinations thereof.

In one embodiment, the compositions or implants of the present invention can deliver bioactive agent, a large molecular weight drug, such as, aflibercept, pegaptanib, or an antibody therapeutic, such as ranibizumab, bevacizumab, trastuzumab, rituximab, gentuzumab, ozagamicin or cetuximab. In some embodiment, the M_{w} of the therapeutic agent is greater than about 10 kDa, about 30 kDa, about 50 kDa, about 75 kDa, about 100 kDa, about 150 kDa, about 200 kDa.

In one embodiment, the disease, or disorder is pain, inflammation, cataracts, allergies, age-related macular degeneration (AMD), diabetic retinopathy (DR), macular edema, diabetic macular edema (DME), cytomegalovirus (CMV), retinitis, retinitis pigmentosa, uveitis, dry-eye syndrome, keratitis, glaucoma, blepharitis, blephariconjunctivtis, ocular hypertension, conjunctivitis, cystinosis, vitreomacular adhesion, corneal neovascularisation, corneal ulcers and post-surgical ocular inflammations/wound healing.

### EXAMPLES

### Materials

The following methods and materials were used in the Examples below

Poly(lactic-co-glycolic acid) (PLGA) 5002A (50% lactic acid, 50% glycolic acid monomers) and PLGA 7502A (75% lactic acid, 25% glycolic acid) (referred to as PLGA50/50 and PLGA75/25 respectively throughout) was purchased from Corbion Purac Biomaterials (Gorinchem, The Netherlands). Poly(ethylene glycol) diacrylate (PEGDA) molecular weight (M_{w}) 258, 575 and 700 Da, ovalbumin (OVA), bovine serum albumin (BSA), Irgacure 2959 (1-[4-(2-hydroxyethoxy)-phenyl]-2-hydroxy-2-methyl-1-propanone), methanol (HPLC grade) and acetonitrile (ACN) (HPLC grade) were purchased from Sigma-Aldrich (Dorset, United Kingdom). Triamcinolone acetonide (TA) and dexamethasone (DEX) was purchased from Spruyt Hillen by (Ijsselstein, The Netherlands). Bevacizumab (BVZ) (Avastin®) was purchased from local pharmacy (manufactured by Roche, Switzerland; each vial consists of 100 mg BVZ in 4 mL i.e, 25 mg/mi). Fluorescein isothiocyanate-dextran (FITC-Dextran) (M_{w} 150 kDa) was purchased from TdB Consultancy AB (Uppsala, Sweden). 27G needles and 1 ml syringes were purchased from Terumo Europe N.V. (Interleuvenlaan, Belgium). Rabbit anti-OVA-biotin conjugate (polyclonal) was purchased from Novus Biologicals (Cambridge, United Kingdom). Streptavidin-Horse radish peroxidase conjugate was purchased from BioLegend® (San Diego, United States). Superblock T20 buffer was purchased from Thermo Scientific Pierce (Rockford, United States).

### In Situ Photocrosslinked Implants (ISPcl)

### Example 1, Preparation of ISPcl Gel Formulations

For preparation of ISPcl, the molecules under investigation (DEX, OVA, BSA, FITC-dextran 150 kDa and BVZ) were firstly added in selected PEGDA (M_{w}=700 Da) at a concentration of 0.5% w/w. Once the molecules were fully dissolved or suspended, the desired amount of PLGA 75/25 was then added to the molecule/PEGDA mixture and left to dissolve at room temperature to produce 30% w/w PLGA formulations. Prior to photocrosslinking predetermined amount of photoinitiator Irgacure® 2959 (2% w/v in 70% ethanol in water as stock solution) was added to the formulation and vortexed for predetermined time to ensure complete mixing.

### Example 2, In Vitro Drug Release Study

For drug release studies selected ISPcl gel formulation were injected (approx. 0.2 g or 0.1 g) into desired amount of PBS (pH 7.3±0.2). Sink conditions were maintained for each drug type. ISPcl were formed by exposing them immediately to 365 nm using bench-top UV light (at 3.1±0.1 mW/cm², CAMAG, Muttenz, Switzerland) for varying periods of time in PBS. The implants were stored in an incubator (37° C. and 60 rpm) for the duration of the release study. At predetermined time intervals the entire PBS medium was removed and replaced with equal amount of fresh PBS. All the experiments were carried out in triplicates. The concentration of released drug molecule in the PBS samples was analyzed as described below.

Analysis of DEX and TA samples were carried out using reversed-phase HPLC with UV detection (Agilent 1260 Infinity Quaternary System) using an Agilent Zorbax Eclipse Plus 250 mm C18 column (250 mm length, 4.6 mm internal diameter and 5 μm particle size) and an Agilent Zorbax guard column held at 25° C. (Agilent Technologies UK Ltd, Stockport, UK). Analysis required a mobile phase of 60% water and 40% acetonitrile with UV absorbance at 270 nm (for DEX) and 236 nm (for TA) at a flow rate of 1 ml/min and 0.8 ml/min respectively. Analysis of FITC-Dextran 150 kDa release was conducted using a fluorescence spectrophotometer. To a black 96-well plate, 150 μL of FITC-dextran 150 kDa sample was pipetted. The plate was then analysed using a BMG Labtech FLUOstar Optima fluorescence plate reader (BMG Labtech GMBH, Ortenberg, Germany). Fluorescence excitation occurred at 480 nm with emission measured at 520 nm. The gain was set at 828 and the plate was read at 37° C. Fluorescence values were collected and examined using BMG Labtech OPTIMA software (version 2.20). BSA, OVA and bevacizumab (Avastin) were assayed using a Pierce™ Micro BCA protein assay kit (Thermo Scientific, Hampton, UK). To a microwell, 150 μL of BSA, OVA or BVZ standard or release sample was pipetted and 150 μL of working reagent was added. A plate shaker ensured thorough mixing. The 96-well plate was covered and incubated for 2 hours at 37° C. Once the plate was allowed to return to room temperature, a UV plate reader measured the absorbance at 562 nm. The average absorbance reading of the blank was subtracted from those of the standards/samples. ELISA test for determining the bioactivity of OVA was tested as per our in house protocol.

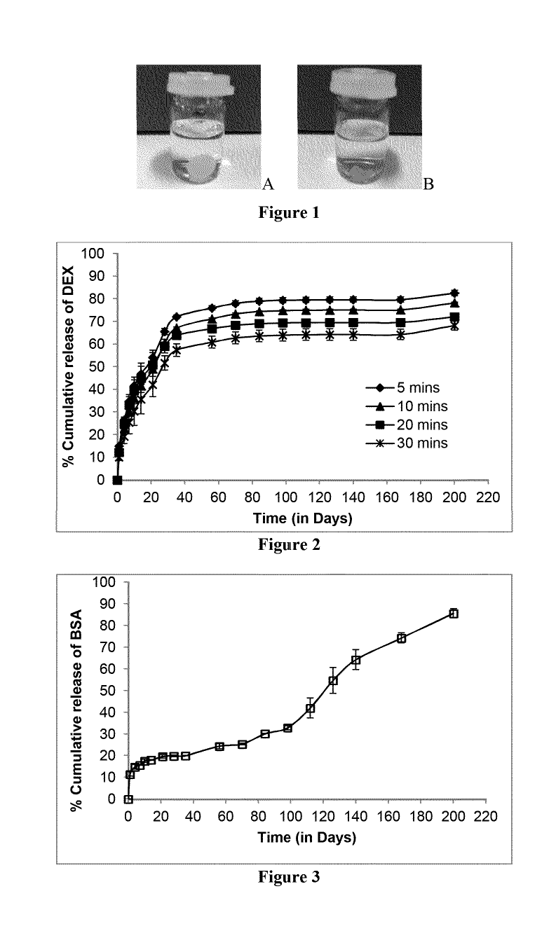

FIG. 1A clearly demonstrates that the ISPcl forms an implant when injected into aqueous environment and subjected to UV light. FIG. 1B indicates that the implants degrade over time. They are numerous factors that govern the extent and rate of drug release and/or biodegradation of the implants, factors for example, polymeric composition, polymer M_{w}, drug type and loading, implant size, time and extent of UV crosslinking and amount and type/concentration of photoinitiator will determine the rate and extent of drug release. The ability to vary these factors also means that implants can be easily tailored to produce desired period of drug release to address specific clinical/patient needs in treating various ocular diseases, this is clearly demonstrated in FIGS. 2-5.

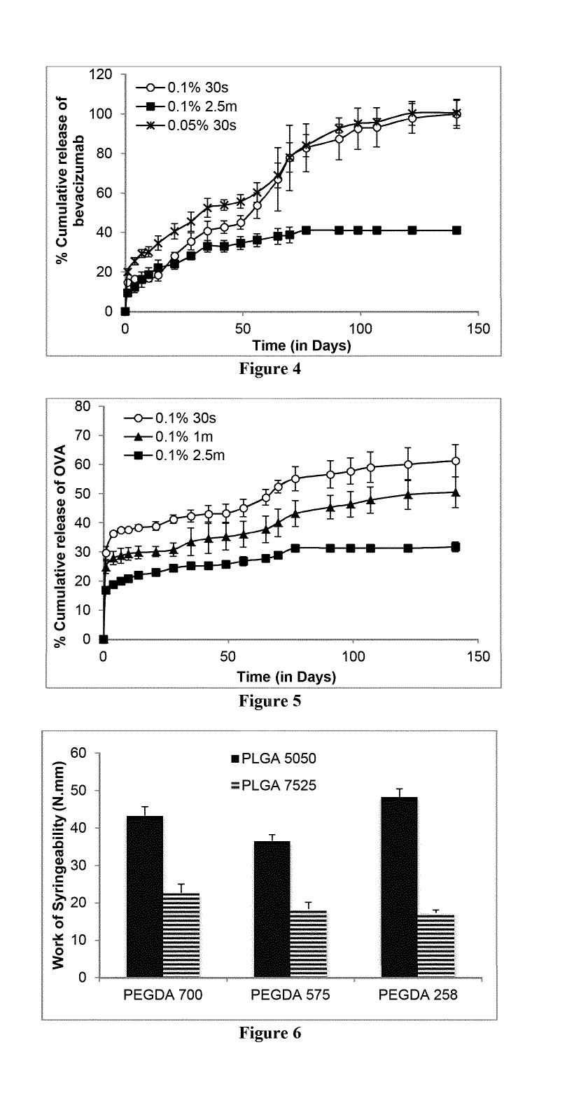

FIG. 2-5 demonstrates in vitro release profiles of various drug molecules from ISPcl of different compositions and crosslinking times. FIG. 2 shows that the percentage release of DEX is dependent upon the time of UV crosslinking, where increase in crosslinking time caused decrease in percentage drug release. For example, after nearly 140 days the mean percentage DEX release was 79.62, 75.15, 69.59 and 64.21 from ISPcl crosslinked for 5, 10, 15 and 30 min, respectively. In all cases low burst release (<15%) is noted, which is also dependent upon the time of UV crosslinking. In addition to small molecules, the mainstay of treatment of PS diseases such as AMD, DR and DME treatment are anti-VEGF therapies such as bevacizumab (Avastin®) and ranibizumab (Lucentis®), it was paramount to investigate the ability of these ISPcls to deliver large molecular weight biologics molecules for long-term, as these drugs are injected indefinitely in the eye on monthly basis. Therefore, we have investigated release of model protein molecule, BSA and OVA and anti-VEGF molecule bevacizumab (BVZ). BSA has a M_{w} of 66 kDa. OVA have a M_{w} of 45 kDa which is nearly similar in M_{w} to commercially available anti-VEGF drug ranibizumab (Lucentis®). BVZ has a high M_{w} of 149 kDa. FIG. 3 shows long-term controlled release of BSA from 30% PLGA/69.4% w/w PEGDA700 crosslinked implants for 5 min, with nearly 86% of BSA released after 200 days. Likewise, FIGS. 4 and 5 shows controlled release of BVZ and OVA from the ISPcl implants. It is evident that either by varying the concentration of the photoinitiator or crosslinking time the amount of drug released can be controlled, where higher crosslinking time or higher concentration of photoinitiator can sustain the drug release for longer periods of time. For example, approximately 41% and 100% BVZ was released from 30% PLGA ISPcl implants UV crosslinked for 2.5 min and 30 sec, respectively. Similar trend was noted with regards to OVA release from ISPcl. It is clear from the FIGS. 4 and 5, that the drug release can be sustained for a period >140 days, by varying the crosslinking time. Furthermore, unlike PLGA only implants, which has short degradation time (e.g. 50-60 days for PLGA 50/50 and 3-4 months for PLGA 75/25 alone), drug release ISPcls can be considerably extended due to cross-linked nature of the implant—where controlled drug release for a period of greater than 200 days (>6 months) as been demonstrated (FIG. 2-3), which can be varied by varying the degree of crosslinking.

Due to slow degradation rate of the crosslinked ISPcls, the drastic drop in local pH is further delayed (unlike PLGA only implants) which is especially important and an advantage in protecting the sensitive molecules such as peptides and proteins. We have demonstrated that our novel ISPcl systems are stable and avoid protein degradation. For example, an ELISA test was conducted to determine the bioactivity of released OVA from the ISPcl, at 37±2° C., after 1 and 3 months, respectively. Nearly 97±2% of the OVA remained active as demonstrated by the ELISA, which clearly indicates excellent stability of protein molecules in our delivery systems. This clearly indicates that the PLGA/PEGDA implants not only sustain the release of protein molecules but it also matins protein activity.

### Example 3, Syringeability of ISPcl

Syringeability is a very important parameter in considering whether a formulation is suitable to be delivered via a syringe and needle, especially if the needle in question has a small bore, as would be required for ocular delivery. Therefore, Work of Syringeability (WoS) was investigated to determine the effort that would be required to expel the ISPcl gel formulations through 27G needle that is commonly used in intraocular injections. Briefly, 1 ml disposable medical syringes (Becton, Dickinson and Company, Oxford, UK) were filled with the ISPcl gel formulations to a constant height equivalent to 0.1 ml. Using the Texture Analyser (Stable Micro Systems, Surrey, UK), the content of the syringe was expelled at a rate of 0.5 mm/second. The area under the resultant force-distance plot was used to determine the WoS using the Exponent TA.XT software (Version 4.0). The WoS observed relating to expelling air from a blank syringe was subtracted from the experimental results to ensure the data collected related solely to the formulation under study. An increase in WoS was conveyed by an increase in the area under the curve. All measurements were performed in at least triplicate.

In addition to drug release, it is also important to demonstrate the injectability of these in situ forming implant gels, as these are designed to be injected in the eye using hypodermic needles or microneedles following short-term application of UV light. FIG. 6 represents the WoS for each ISPcl formulation that was calculated from the resulting force-distance plots of Texture-Analysis. The WoS data indicates that the PLGA/PEGDA formulations for both PLGA 50/50 and PLGA 75/25 require different forces to expel them from the syringe with 27G needle. In general the PLGA75/25 formulations are more easily expelled compared to the PLGA50/50 formulations with a WoS of 43.23 N·mm calculated for the PLGA50/50-PEGDA700 formulation, with 22.55 N·mm calculated for the PLGA75/25-PEGDA700 formulation. It would be expected that the highest molecular weight of PEGDA would result in the greatest resistance to expulsion. This trend is followed when considering the PLGA75/25 formulations but not with the PLGA50/50. The greatest WoS was seen with the PLGA 50/50-PEGDA258 formulation, 48.24 N·mm, which is significantly greater than the other PLGA5050 formulations (p<0.0001). Therefore, the implant forming gels can be injected and the forces for injections vary by changing the composition/concentration of the polymers within the ISPcl formulation.

### Example 4, Preformed Photocrosslinked Implants (PPcl)

Similar to ISPcl the molecule/drug under investigation BSA, TA, OVA, and FITC-dextran 150 kDa was firstly dissolved/suspended in PEGDA at different concentrations. Following which desired amount of PLGA 75/25 or 50/50 was added to the drug/PEGDA mixture and left for mixing at room temperature to form a homogenous gels. Finally, desired amount of a photoinitiator Irgacure® 2959 (2% w/v in 70% ethanol in water) was added to the formulation and vortexed for 1 minute to ensure complete mixing. These gels were then casted into molds to form thin films (10×5×0.5 mm) and subjected to photocrosslinking using Fusion UV LightHammer 6 high power UV curing system (Maryland, USA) fitted with a “D” class mercury discharge bulb (270 W/10 nm), with a belt speed of 10 m/min and at wavelength of 365 nm, at different lamp intensities (LI) and for different cycles/runs (the expose time for each run is 3.4 sec) to form PPcl. In vitro drug release studies were conducted as given for ISPcl in PBS. Drug samples were collected at a predetermined time intervals and analyzed using the techniques as stated above.

In addition to using the ISPcl as an injectable implant forming ocular delivery system, the PLGA/PEGDA composition invented here can also be used as preformed implants. This offers additional opportunity for our delivery systems where the preformed photocrosslinked implants (PPcl) can be simply inserted in the eye (e.g. in the fornix or subconjunctively) to treat the front of the eye diseases or can also be administered (e.g. intravitreally) in the eye, using an applicator, for treating the back of the eye diseases. The PPcl are made as detailed above, FIG. 8 shows a digital and SEM image of these implants. These implants can be fabricated in a variety of shapes (e.g. rods, films, cylindrical or circular) and sizes, including in the form of micro- or nanoparticles.

Using PPcls we have demonstrated controlled release of BSA, FITC-Dextran 150 kDa, and TA over different periods of time, as shown in the FIG. 9-12. FITC-Dextran 150 kDa was selected, as its M_{w} is nearly similar to that of commercially available anti-VEGF drug BVZ (Avastin®). As shown in FIG. 9, the percentage release was dependent upon the M_{w}, where BSA showed higher percentage of release when compared to FITC-Dextran 150 kDa over the period of 266 days. For example, % release of BSA and FITC-Dextran 150 kDa was approx. 72 and 27%, respectively after 266 days, which is predicted to continue for another few months. This is due to the fact that the BSA molecule is nearly 2.27-times smaller in M_{w} than FITC-Dextran 150 kDa. Here, we have seen nearly zero-order release of the molecules with no burst release when compared to the ISPcls, this is due to the high crosslink density or tight network structure of the PPcl that significantly slows drug diffusion, when compared to the ISPcls. Furthermore, like the ISPcl, the PPcls are also biodegradable however the rate of degradation is slower has compared to ISPcl. Furthermore, the PPcls can be fabricated to have a single and/or multiple layers, which will enable loading of more than one or more drug molecule or same drug with different release profiles or rates.

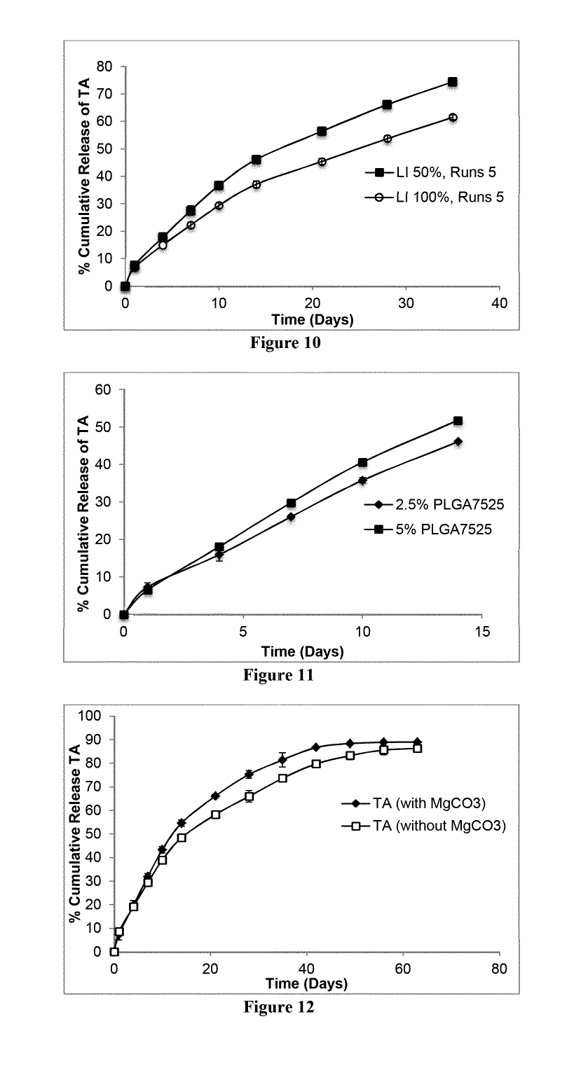

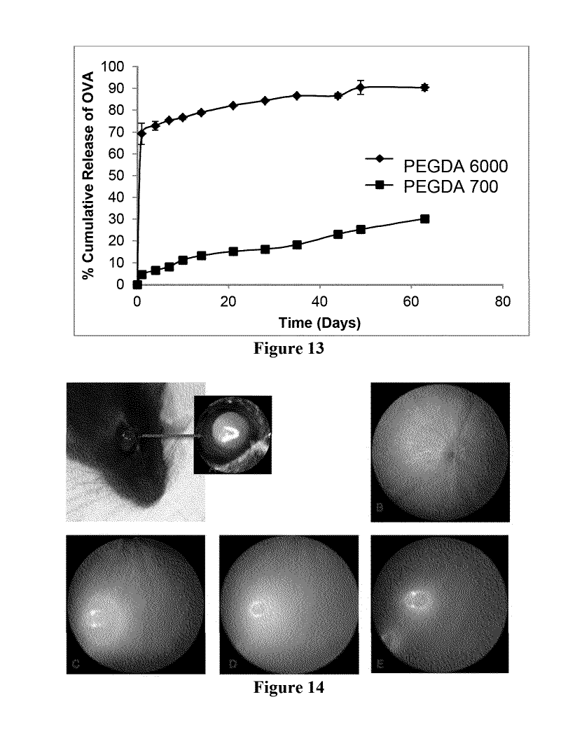

Unlike long-term drug release, drug release for short-term is also advantageous in treating certain common ocular diseases of the front of the eye (e.g. glaucoma, dry-eye syndrome, keratitis, blepharitis, and other types of bacterial/fungal inflammations) that require short-term drug delivery. In this regard FIG. 10-13 shows short-term release of a small molecule, TA, where the TA release can be for 2 weeks to 9 weeks. Particularly, when PPcl were fabricated at IL of 50 and 100%, with 5 runs, nearly 75 and 62% of TA was released within 35 days (FIG. 10). Likewise, when a low level of PLGA was used nearly 46% of TA was released within 14 days, which increased to 52% with decrease in PLGA concentration (FIG. 11). Furthermore, by adding a pore-forming agent (e.g. MgCO₃), the amount of drug (TA) released from the implant can be increased. Without wishing to be bound by theory it is believed that an increase in pore density of the implant will allow higher drug release (FIG. 12). On the other hand, when the M_{w} of PEGDA was varied from 700 Da to 6000 Da in the preparation of PPcls, TA release increased from 11% to 77% after 10 days (FIG. 13). This is due to the fact that the PPcl with low M_{w} PEGDA has higher crosslink density when compared to high M_{w} PEGDA. Overall, these invention suggests that by simply varying the composition and/or time of crosslinking of the PPcls the drug release can be easily tailored, thereby providing greater degree of flexibility in designing these implants that can be utilized to address specific ocular diseases, where either short-term to long-term drug release is required.

### Example 5, Biocompatability of Polymeric Matrix

In order to gain cytotoxicity data of polymer matrix utilized in preparation of ISPcl and PPcl, the materials were exposed to human retinal epithelial cells line (ARPE-19). For this a MTT (3-(4,5-dimethylthiazol-2-yl)2,5-diphenyl tetrazolium bromide) cleavage assay was used that was originally described by Mosmann for measuring cell survival/proliferation¹². The assay detects live cells therefore this method can be used to measure cytotoxicity of materials. The MTS assay is often described as a ‘one-step MTT assay’ as it allows the addition of the reagent straight to the cells without the intermittent steps that are required with the MTT assay. MTS (3-(4,5-dimethylthiazol-2-yl)-5-(3-carboxymethoxyphenyl)-2-(4-sulfophenyl)-2H-tetrazolium), in the presence of phenazine methosulfate (PMS), produces a formazan product that has an absorbance maximum at 490-500 nm.

The PLGA/PEGDA formulation was produced in a sterile environment with the PEGDA being filtered through a sterile 0.2 μm syringe filter (VWR®, International Ltd, Leicestershire, UK). The ISPcl formulation (0.1 g) was injected in 5 ml of DMEM/F-12 media (Gibco®, Life Technologies™, Paisley, UK) in autoclaved glass vials. The formulation was subjected to UV crosslinking at 365 nm, similar to in vitro release studies and at predetermined time points (1, 30 and 120 days after formation), the entire release media was collected, stored and replaced with fresh media. The collected media was then subjected to cytotoxicity studies. All treatments were performed on ARPE-19 cells seeded in 96-well plates (Nunc®, Denmark) at a cellular density of 1.75×10⁴ cells/well, which were incubated at 37° C. for 24 h in DMEM/F-12. The DMEM/F-12 medium was removed and replaced with 200 μl of the release media from each time point (fresh media was used as the control). Subsequently, the cells were incubated for a further 24 hours. Cell viability was determined using the cell proliferation assay where 20 μl of Promega G3580 MTS assay solution (Promega Corporation, Wisconsin, USA) was added to each well. After 2 hours of incubation, the UV absorbance was determined at 490 nm.

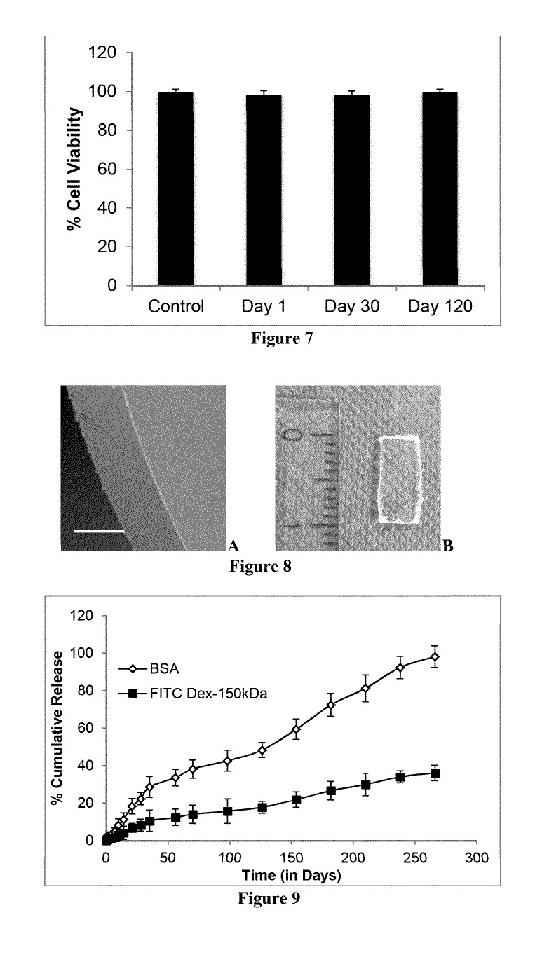

We have also demonstrated that the ISPcls are biocompatible in nature, as presented in FIG. 7. When the release samples of the implants was exposed to human retinal pigment epithelial cell lines (ARPE-19) over different time periods, it showed compatibility nearly similar to that of the control samples (culture media), therefore, indicating biocompatibility with ocular cell lines.

### Example 6, In Vivo Implant Formation

2 μl of ISPcl gel formulation was injected by intravitreal route in the rat eye, following by UV light exposure for 2 min, fundus images were collected to locate the implant formation and image model dye release within the eye. Likewise PPcl were administered by subconjunctival route and any surface inflammation monitored by experienced ophthalmologist

In in vivo experiments we have demonstrated that the ISPcls form an implant upon injection in the eye (intravitreal route) and biodegradation over time (FIG. 14). A fluorescent molecule was used in this study to show that the drug release occurs over time and implant degradation without causing any damage to the ocular tissues, as seen by fundus imaging. This indicates that this polymeric composition is biocompatible to ocular tissues and therefore considered safe for application in the eye.

1. An ocular composition comprising:
99 to 60% (w/w) of a photopolymerizable composition, the photopolymerizable composition being selected from the group consisting of one of fragments and monomers of polyalkylene glycol diacrylate, one of fragments and monomers of polyalkylene glycol dimethacrylate; mixtures thereof; copolymers thereof; and block copolymers thereof,
the photopolymerizable composition having a molecular weight in a range of 100 to 20,000 Dalton;
a biodegradable polymer selected from the group consisting of lactide/glycolide co-polymer (PLGA), polylactic acid (PLA), polyglycolic acid (PGA), polycaprolactone (PCL), lactide/caprolactone copolymer (PLC), poly (L-lactide) (PLLA) mixtures thereof, copolymers thereof, and block copolymers thereof;
a photoinitiator; and
a therapeutic agent.

2. The ocular composition of claim 1, wherein the ocular composition is used to at least one of form an ocular implant and coat an ocular implant.

3. The ocular composition of claim 2, wherein the ocular implant is an in situ formed ocular implant.

4. The ocular composition of claim 3, wherein the photopolymerizable composition has a molecular weight in a range of 100 to 6,000 Dalton.

5. (canceled)

6. (canceled)

7. The ocular composition of claim 2, wherein the ocular implant is a pre-formed ocular implant.

8. (canceled)

9. (canceled)

10. The ocular composition of claim 1, wherein the photopolymerizable composition is one of a polyalkylene glycol diacrylate fragment and a polyalkylene glycol diacrylate monomer, the photopolymerizable composition including diacrylate end units, the diacrylate end units being selected from the group consisting of:
one of fragments and monomers of polyether;
one of fragments and monomers of polyester;
one of fragments and monomers of polycarbonate;
mixtures thereof;
copolymers thereof; and
block copolymers thereof.

11. The ocular composition of claim 1, wherein the photopolymerizable composition is selected from the group consisting of polyethylene glycol diacrylate, diethylene glycol diacrylate, polyethylene glycol dimethacrylate, diethylene glycol dimethacrylate, polypropylene glycol diacrylate, dipropylene glycol diacrylate, dipropylene glycol dimethacrylate, and polypropylene glycol dimethacrylate.

12. (canceled)

13. (canceled)

14. The ocular composition of claim 12, wherein the biodegradable polymer is PLGA.

15. The ocular composition of claim 14, wherein the PLGA includes a molar ratio of lactic acid to glycolic acid, the molar ratio of lactic acid to glycolic acid in the PLGA being one of 90% lactic acid to 10% glycolic acid, 85% lactic acid to 15% glycolic acid, 75% lactic acid to 25% glycolic acid, 65% lactic acid to 35% glycolic acid, 50% lactic acid to 50% glycolic acid, 35% lactic acid to 65% glycolic acid, 25% lactic acid to 75% glycolic acid, 15% lactic acid to 85% glycolic acid, and 10% lactic acid to 90% glycolic acid.

16. The ocular composition of claim 15, wherein:
the photopolymerizable composition is 79.5 to 59.5% (w/w) of one of polyethylene glycol diacrylate and polyethylenene glycol dimethacrylate; and
the biodegradable polymer is 20 to 40% (w/w) PLGA, the molar ratio of lactic acid to glycolic acid in the PLGA being at least one of 90% lactic acid to 10% glycolic acid, 85% lactic acid to 15% glycolic acid, 75% lactic acid to 25% glycolic acid, and 50% lactic acid to 50% glycolic acid.

17. (canceled)

18. The ocular composition of claim 1, wherein:
a. the photopolymerizable composition is 95.5 to 84.5% (w/w) of one of polyalkylene glycol diacrylate and polyalkylene glycol dimethacrylate; and
b. the biodegradable polymer is 4 to 15% (w/w) PCL.

19. The ocular composition of claim 1, wherein:
a. the photopolymerizable composition is 79.5 to 94.5% (w/w) of one of polyalkylene glycol diacrylate and polyalkylene glycol dimethacrylate; and
b. the biodegradable polymer is 5 to 20% (w/w) PLLA.

20. The ocular composition of claim 1, wherein:
a. the photopolymerizable composition is 95.5 to 84.5% (w/w) of one of polyalkylene glycol diacrylate and polyalkylene glycol dimethacrylate; and
b. the biodegradable polymer is 4 to 15% (w/w) PLC, the PLC having a ratio of lactic acid to caprolactone, the ratio of lactic acid to caprolactone being one of 90% lactic acid to 10% caprolactone, 80% lactic acid to 20% caprolactone, 70% lactic acid to 30% caprolactone, 60% lactic acid to 40% caprolactone, and 50% lactic acid to 50% caprolactone.

21. The ocular composition of claim 1, further comprising a solvent selected from the group consisting of dimethyl sulfoxide, decylmethyl sulfoxide, 2-pyrrolidone, 1-methyl-2-pyrrolidne, N-vinyl-pyrrolidine, N-Methyl-2-pyrrolidone, N-ethyl-pyrrolidone, glycerol formal, glycerol, polyethylene glycol, propylene glycol, benzyl alcohol, benzyl benzoate, ethyl benzoate, triacetin, triethyl citrate, dimethylformamide, dimethylacetamide and tetrahydrofuran.

22. (canceled)

23. The ocular composition of claim 1, further comprising a pore-forming agent.

24. The ocular composition of claim 23, wherein the pore-forming agent is selected from the group consisting of polyethylene glycol, maltose, glucose, agarose, mannitol, gelatin, sodium chloride, magnesium carbonate, magnesium hydroxide, potassium chloride, sodium bicarbonate, potassium bicarbonate, and sucrose.

25. The ocular composition of claim 1, wherein the photopolymerizable composition is polymerized by irradiating the ocular composition with light at a wavelength of at least one of between 230 to 550 nm, between 300 to 525 nm, and between 350 to 490 nm, the ocular composition being irradiated for between 1 second and 60 minutes.

26. The ocular composition of claim 1, wherein the biodegradable polymer is contained within a matrix of the photopolymerizable composition.

27. The ocular composition of claim 1, wherein the photoinitiator is at least one of a hydroxyketone photoinitiator, an amino ketone photoinitiator, a hydroxy ketone/benzophenone photoinitiator, a benzyldimethyl ketal photoinitiator, a phenylglyoxylate photoinitiator, an acyl phosphine oxide photoinitiator, an acyl phosphine oxide/alpha hydroxy ketone photoinitiator, a benzophenone photoinitiator, a ribityl isoalloxazine photoinitiator, a phenylglyoxylate photoinitiator 1-[4-(2-hydroxyethoxy)-phenyl]-2-hydroxy-2-methyl-1-propanone, 2,2-dimethoxy-2-phenylacetophenone (DMPA), and riboflavin.

28. (canceled)

29. The ocular composition of claim 1, further comprising a co-initiator.

30. The ocular composition of claim 29, wherein the photoinitiator is riboflavin and the co-initiator is L-arginine.

31. The ocular composition of claim 1, wherein the ocular composition is one of a nanoparticle and a microparticle ocular implant.

32. The ocular composition of claim 31, wherein the nanoparticle ocular implant is less than 1,000 nm and the microparticle ocular implant is less than 1,000 μm.

33. (canceled)

34. A method of making an ocular composition, the method comprising the steps of:
mixing a therapeutic agent, a photopolymerizable composition, a biodegradable polymer and a photoinitiator, in any order of addition, to form a mixture;
administering the mixture to an ocular area of a subject; and
irradiating the mixture for between 1 second and 60 minutes with light at a wavelength of at least one of between 230 to 550 nm, between 300 to 525 nm, and between 350 to 490 nm to form the ocular composition.

35. (canceled)

36. (canceled)

37. The method of claim 36, wherein the irradiating step is with light at a wavelength of at least one from the group consisting of 365 nm and 475 nm for at least one from the group consisting of 1 second, 2 minutes, 5 minutes, 10 minutes, 20 minutes, and 30 minutes.

38. A method of making an ocular composition, the ocular composition being one of a nanoparticle ocular implant and a microparticle ocular implant, the method comprising the steps of:
mixing a therapeutic agent, a photopolymerizable composition, a biodegradable polymer and a photoinitiator, in any order of addition, to form a first mixture, the photopolymerizable composition having a molecular weight of between 100 and 20,000 Dalton;
adding the first mixture to an aqueous medium to form a second mixture;
sonicating the second mixture; and
irradiating the second mixture for between 1 second and 60 minutes with light at a wavelength of one of between 230 to 550 nm, between 300 to 525 nm, and between 350 to 490 nm to form one of nanoparticles or microparticles.
